(19) United States
(12) Patent Application Publication
(10) Pub. No.: US 2003/0054333 A1
(43) Pub. Date: Mar. 20, 2003

(54) Biosensor for use in toxicity assessment and pharmacological screening

(76) Inventors: James J. Hickman, Pendleton, SC (US); Douglas A. Kirkpatrick, Great Falls, VA (US); David A. Stenger, Springfield, VA (US)

Correspondence Address: Nisan A. Steinberg, Esq., SIDLEY AUSTIN BROWN & WOOD LLP, 555 West Fifth Street, Los Angeles, CA, 90013, US

(21) Appl. No.: 09/880,138

(22) Filed: Jun. 12, 2001

Related U.S. Application Data
(63) Continuation of application No. 09/513,720, filed on Feb. 24, 2000, now abandoned.
Application No. 09/513,720 is a continuation of application No. 09/372,568, filed on Aug. 11, 1999, now abandoned.
Application No. 09/372,568 is a continuation of application No. 09/236,684, filed on Jan. 25, 1999, now abandoned.
Application No. 09/236,684 is a continuation of application No. 09/109,481, filed on Jul. 2, 1998, now abandoned.
Application No. 09/109,481 is a continuation of application No. 08/912,033, filed on Aug. 15, 1997, now abandoned.
(60) Provisional application No. 60/023,413, filed on Aug. 16, 1996.

(51) Int. Cl. C12Q 1/00; C12M 1/34; C12N 5/08

(52) U.S. Cl. 435/4; 435/287.1; 435/368

(57) ABSTRACT

A biosensor for detecting a bioeffecting substance in a test sample includes a cell network of at least one electrically excitable cell provided on a substrate in a predetermined geometry with a predefined axonal/dendritic polarity. The at least one cell is capable of producing a signal in response to the presence of the bioeffecting substance. At least one signal transducer is provided with the substrate in a predetermined geometry and is capable of detecting the signal produced by the cell. A culture medium capable of supporting metabolism of the cell is also provided.

### GOVERNMENT RIGHTS

The present invention was performed in part by employees of the National Research Laboratory. The Government may have certain rights in the invention.

### 1. FIELD OF THE INVENTION

The present invention relates to a biological sensor useful in the detection of bioeffecting environmental conditions. The biosensor can be used to test for the presence of toxic substances and for screening drugs for pharmacological benefit.

### 2. BACKGROUND OF THE INVENTION

Programs that monitor environmentally hazardous toxins would benefit from sensors based on cell-cell communication, which is one of the lowest levels of cognitive function. One of the reasons they have not been developed to date is the wide range of expertise needed for their reduction to practice. An example of such an application is to assess the effect of waste sources and waste products that may be unknown, especially minor components, which have not been fully identified and characterized. A sensor based on cell-cell communication could screen a processed waste stream to detect toxins or material without requiring that a specific agent be known. Such a biosensor would ensure the safety of communities near bioremediation sites. This type of sensor would also be useful to screen compounds for toxic effects in pharmaceutical or other manufacturing situations, as well as for discovering useful effects of those compounds for drug discovery applications.

2.1. Other Sensors

The types of sensors currently in use or under development include ion-sensitive electrodes (Solsky, 1990), sensors based on antibody binding (Aizawa, 1991), living cells (Parce et al., 1989; Leech & Rechnitz, 1993), and others (Hughes et al., 1991; Edelman & Wang, 1991; Blum & Coulet, 1991). Most sensors rely on the fact that the agent to be tested is known and the sensors are thus targeted for the specific known agent. Additional information beyond this is currently obtained primarily from epidemiological studies on animals or humans and requires a full scale biochemistry lab staffed by highly trained professionals. It would be particularly beneficial to develop sensors to function as precursors to these more expensive and time consuming types of studies, particularly for toxic substances that are not currently recognized or are present in minute quantities in a test sample.

In vitro cell culture of embryonic rat tissue has also been employed in an attempt to study cellular organization and communication. However, in the studies to date, neurons in culture are disorganized in the sense that the connections between cells are not controllable and therefore not reproducible. Historically, this has made it difficult to relate specific signals to specific functions as well as provide for the reproducible system of neuron interconnection necessary to measure small changes in neuron activity associated with changes in the environmental condition of the cells.

One type of sensor that is beginning to address the need for sensors based on general biological function is the Light Addressable Potentiometric Sensor (LAPS) biosensor, a whole cell physiometer that measures metabolic rate. However, sensors that can detect unknown agents or agents of intermediate toxicity that affect motor function, cognitive function, and/or other higher order processes are primitive or do not exist.

2.2. Bioeffecting Substances, Environmental Toxins, and Pharmaceutical Agents.

There are potentially a large number of bioeffecting substances, including toxins, of which only a handful are known for their bioeffecting characteristics. It would thus be useful to determine whether a sample contains a bioeffecting substance of a known type or, more importantly, an unknown type. Of particular importance in hazardous waste remediation is the detection of toxins, such as neurotoxins, in a test sample.

Biosensors according to the present invention allow for the detection of these and other bioeffecting substances, whether for their harmful or beneficial effects as a precursor to animal testing. This can have great impact on pharmaceutical toxin screening and drug discovery as well.

2.3. Fabrication of Geometric Patterns

The fabrication of geometric patterns on surfaces for biological applications has involved etched grooves, adaptations of conventional photoresist technology, plasma modification, Langmuir-Blodgett films, and metal evaporation.

Cell patterning has also been demonstrated through the use of topographical strategies that do not rely on spatially controlled cell adhesion. For example, Curtis et al. (Curtis and Varde 1964; Curtis and Clark 1990); Curtis, Breckenridge et al. 1992) have shown that etched substrates can be used to control the extension of neural cell processes within topographical barriers. In this case, however, arbitrary control of axonal/dendritic polarity within topological barriers has not been demonstrated.

Prior methods have not allowed one to hypothesize that a microlithographic approach might be taken to geometrically predispose neurons to send one or more process in a preferred direction. At best, microlithographic approaches to patterning the adhesion of neuronal cells have been investigated. A variety of low resolution techniques have been established to control the adhesion of cultured cells (Ivanova and Margolis 1973; Cooper, Munden et al. 1976; Albrecht-Buehler 1980; Albrecht-Buehler 1980; Cooper 1976; Ivanova 1973; Hammarback 1985; Hammarback 1988; Hammarback 1986). These approaches have failed to demonstrate the production of isolated features having dimensions in the 1 to 5 micron range, much less establish control over axonal/dendritic cell polarity. Thus, there has been no report of an effective solution to the problem of controlling the orientation of axonal growth on synthetic surfaces in vitro.

Recently, improved methods for defining the axonal/dendritic polarity of cells and/or networks of cells have been developed. These methods are the subject of U.S. Ser. No. 08/689,970.

2.4. Techniques for High Resolution Substrate Design

Only recently has photolithography using deep UV irradiation been employed to create monolayer patterns. For instance, U.S. Pat. No. 5,324,591 describes such a system. This technique involves a two-step process whereby a homogeneous self-assembled monolayer (SAM) is irradiated through a mask to create renewed areas of reactivity, which are then rederivatized with a different SAM-forming molecule.

Recently, this method has been optimized through the use of a beam homogenizer, which now permits the routine production of reproducible, high resolution (2-20 micron) organosilane patterns (unpublished work of one of the inventors). The patterns were produced using 193 nm ArF excimer laser radiation through a photomask applied to a silica surface that had been modified with trimethoxysilylpropyldiethylenetriamine (DETA) or (N-ω-(aminoethyl)-3-aminopropyl-trimethoxysilane (EDA). The UV light removed the DETA or EDA in the exposed areas, and a second cytophobic silane (15F) was reacted with the surface to form the pattern. The patterned surface successfully guided neuronal adhesion and neurite outgrowth creating circuits of embryonic (E18-19) hippocampal neurons in vitro.

Photolithography has been used for cell culture surfaces, with self-assembled monolayers of EDA or DETA for promoting cell adhesion and SAMs of (tridecafluoro-1,1,2,2-tetrahydrooctyl)-1-dimethylchlorosilane (13F) for refracting cell adhesion (Stenger et al. 1992). A new method of preparing the DETA using a non-aqueous preparation gave superior results. In these studies, rat embryonic hippocampal cells selectively adhered to the EDA or DETA monolayer. However, no control of neuronal polarity necessary for a reproducible biosensor was obtained in that system.

There have been several additional reports on the culture of mammalian cells on SAMs. In general, SAMs that are modified with positively charged terminal groups are suitable for cell culture.

Kleinfeld et al. (1988) describes patterned SAM surfaces on silicon and quartz substrates. Patterned growth of cerebellar cells from tissue explants is described as are surfaces of increased and decreased cell attachment prepared by photolithography techniques. However, controlled cell polarity is again not described.

Stenger et al. (1992) describe EDA/13F patterned monolayers used to spatially control the adhesion and direct the outgrowth of rat hippocampal neurons and porcine aortic endothelial cells. The publication indicates that patterns of controlled geometry may provide new approaches for, inter alia, intercellular communication, or to control the alignment of individual cells with transducer elements in biosensors. There is no mention or suggestion, however, of a biosensor having at least one cell and at least one transducer arranged in a configuration so as to monitor changes in an axonal wave potential.

Hickman et al. (1994) investigated the mechanisms involved in the generation of patterned EDA/13F monolayers using deep UV (DUV) lithography. Also disclosed are SAMs of DETA, PEDA, BIPY and PL. The paper states that control of neural outgrowth will allow selection of polarity and orientation of cultured neurons and geometrical control of formation of junctions between cells and their spatial relationship to sensor elements. However, details of such an arrangement are not given.

U.S. Pat. No. 5,391,463 describes UV irradiation of, inter alia, glass and silicon nitride surfaces containing thiols, epoxy groups or vicinal diol groups to result in a surface having reduced adsorption of certain biomolecules, including proteins.

U.S. Pat. No. 5,077,085 describes a substrate having a self-assembling monolayer with a patterned surface having regions of differing reactivity. A catalyst is adsorbed to the modified substrate, followed by metal plating at the adsorbed regions. The technology is described as useful in the manufacture of printed circuits and integrated circuits.

Robinson (Signal, February 1994) briefly describes patterned substrates having diethylenetriamine (DETA) and 13F regions. DETA is described as promoting neuronal survival. The publication states that the dominant cell growth process on DETA is axon development. There is no description of controlled cell polarity or use of multiple transducers to detect axonal wave potentials.

Direct electrical measurement of neuronal impulses have been made for the most part only by the patch-clamp technique. In one deviation from this norm, Fromherz et al. (1991) measured a neuronal cell membrane potential using a neuron-silicon junction. In that case, a neuron was attached to the open gate of a p-channel field effect transistor (FET), and stimulated or spontaneous action potentials directly modulated the source-drain current in the silicon. Such a system requires the placement of a neuron directly on the gate by micromanipulation, thereby being time consuming and open to reproducibility problems. There are no reported biosensors that monitor the axonal wave potential.

U.S. Pat. No. 4,832,759 describes the use of “surface discontinuities” to at least partially define cell adhesion in zones having a width of between 0.2 and 20 microns. U.S. Pat. Nos. 4,591,570 and 4,011,308 describe the use of patterns or arrays of antibody-coated spots for specific immunoabsorption of cells to optically-sensitive surfaces. U.S. Pat. No. 4,562,157 describes the photo-induced activation of adhered chemical species so that chemical functionalities and proteins may be covalently attached to “BIOCHEMFET” devices. However, this work does not address the problem of nonspecific absorption of proteins.

At least one biosensor has been developed which optically measures the metabolic activity of immobilized cells (Parce et al., Science, Vol. 246, p. 243 (1989)). However, groups of cells, not individual cells, are “immobilized” by gravitational sedimentation into micromachined silicon wells.

U.S. patent application Ser. Nos. 07/022,439 filed Mar. 6, 1987 and 07/182,123 filed Apr. 14, 1988 disclose a method for preparing high resolution patterns of metals on solid substrates, by irradiation of an adherent thin film with deep ultraviolet (DUV) irradiation. However, there is no suggestion of patterned substrates for the selective adhesion and outgrowth of cells.

2.5 Previous Approaches to Inducing Directionality of Processes

Corey and co-workers (Wheeler, J. M., et al. in J. Neurosci. Res. (1991) 30:300-307; Corey, J. M., et al., in Soc. Neurosci. Abstr. (1991) 17:210) have published results showing that microlithographic patterns can be created for controlling neuronal morphologies at dimensions smaller than the dimension of the neural cell body. (When freshly plated after dissociation from tissue, hippocampal neurons have cell bodies with a mean diameter of about 8 microns. Upon maturity, the hippocampal neural cell body has a diameter of about 20-25 microns). These workers showed that hippocampal neuron cell bodies could be made to adhere to adhesive, circular somal adhesion sites having a diameter of about 20 microns. These sites are each interconnected by single, 5-micron-wide adherent lines. Cell bodies preferentially adhered and/or migrated to the circular somal adhesion sites instead of the interconnecting adherent lines. The interconnecting adherent lines are apparently too narrow to encourage the adhesion of the cell bodies against a non-adherent background.

The cells, which adhered to the circular somal adhesion sites, are able to extend processes down the interconnecting adherent lines to form a rudimentary type of neural architecture (Wheeler, B. C. and Brewer, G. J., in Enabling Technologies for Cultured Neural Networks, Stenger, D. A. and McKenna, T. M. (Eds.) (Academic Press, San Diego, Calif., 1994) pp. 167-185). These cells, however, did not (and could not be made to) send axons in a preferred or predetermined direction. Thus, the resultant patterns are comprised merely of cell bodies spaced at regular intervals. There is no control over the directionality of the eventual synapse formation and, hence, no control, geometric or otherwise, over axonal/dendritic polarity.

A number of microlithographic templates have been manufactured in the past in an attempt to dictate the geometries of cultured hippocampal neurons. The Gen 1 lithographic mask, described by Stenger et al. 1992, contained simple “chicken wire” features that are used to define crudely the adhesion of hippocampal neurons.

The previously known Gen 2 lithographic mask (Hickman et al., 1994) is used to show that one can preferentially direct the adhesion of cell bodies (soma) and still allow for subsequent neurite outgrowth. Phase contrast micrographs of this mask and immunocytochemical staining, using an axon-specific marker, do not demonstrate the successful induction of axonal/dendritic polarity. Hence, any features disclosed in these prior publications are ineffective in controlling the direction of axonal outgrowth.

It should, thus, be apparent that there remains an unfulfilled need for a biosensor, e.g., cultured neuronal cell biosensor, including without limitation a particular cell-type and substrate design, in which the geometry of axonal/dendritic polarity (and thereby, for example, neuronal network design) can be predetermined.

In summary, previous work fails to teach or suggest a biosensor based on rudimentary cognitive function in which changes in the axonal wave potential are monitored and correlated to the presence or absence of a known or unknown bioeffective environmental condition.

Thus, there exists a need for a biosensor capable of detecting bioeffecting environmental conditions, particularly in the area of toxins. There also remains a need for a method of patterning cell growth wherein cellular polarity is predetermined so as to allow the rational design of neuronal networks for use as the cellular component of biosensors. There is further a need for new types of toxicity sensors that are based on biological function, in particular neuronal function. This need is particularly seen concerning the toxins encountered during pharmaceutical drug screening and for drug discovery.

3.1 SUMMARY OF THE INVENTION

The present invention provides a biological sensor (“biosensor”) for detecting a bioeffecting substance or condition, including toxins and pharmacological agents. Such a biosensor comprises:

(a) a substrate in contact with a culture medium capable of supporting metabolism of one or more electrically excitable cell type;

(b) a cell network composed of at least one viable cell of said cell type, which cell has a predefined axonal/dendritic polarity on said substrate and is capable of producing a signal in response to the bioeffecting substance; and

(c) at least one signal transducer provided proximate said cell network, which is capable of detecting the signal by being operably coupled thereto. The signal detected by said at least one transducer is an action potential or an axonal wave potential.

In a preferred embodiment, a biosensor of the invention further comprises at least one insulating or barrier layer interposed between the above-mentioned transducer(s) and the culture medium, which prevents direct contact between the culture medium and the transducer(s). An insulating layer prevents or impedes the passage of electrons between the cell/culture and the transducer, for example, silica and SiC. A barrier layer, as referred to herein, is an insulating layer that also prevents or impedes the passage of ions between the cell/culture and the transducer, for example, Si₄N₃. The axonal or dendritic polarity is provided by the techniques referred to herein, such as through the use of self-assembling monolayers.

Another embodiment of the present biosensor has no insulating layer and a bare metal electrode is in contact with the cell or close to the cell or its processes. The metal is coated with a SAM or biological macromolecule to create a high impedance seal.

The present invention also contemplates a method of detecting bioeffecting substances in a test sample, which comprises:

(a) providing a test sample and an afore-mentioned biosensor, wherein at least one cell of the biosensor produces a detectable response to at least one bioeffecting substance;

(b) contacting the test sample with the biosensor;

(c) monitoring, with the transducer of the biosensor, a signal produced by the at least one cell in response to contacting it with the test sample; and

(d) correlating the signal to the presence or absence of the bioeffecting substance in the test sample.

The apparatus and methods of the present invention can be used in the construction of bioelectronic circuits for the detection, monitoring, or screening of a wide range of potentially beneficial pharmacological activities and/or harmful substances. Hence, a biosensor is provided for the detection of a substance having biological activity. A preferred biosensor comprises at least one transducer and one or more cells supported by a substrate, which cells are connected in a manner that is based on cognitive function. It should be noted that single neuron can form a circuit with itself. Accordingly, the transducer(s) are sensitive to electrical signals emanating from and travelling between the cells.

A biosensor of the invention can be used for toxicity testing, drug discovery, bioremediation monitoring, bioelectronics, and understanding basic neuroscience.

### 3.2 DESCRIPTION OF THE DRAWINGS

FIG. 1 depicts an exemplary circit model of a biosensor according to the present invention.

FIG. 2A depicts one aspect of the present invention in which neither an insulating layer or barrier is provided between a neuron and a metal electrode.

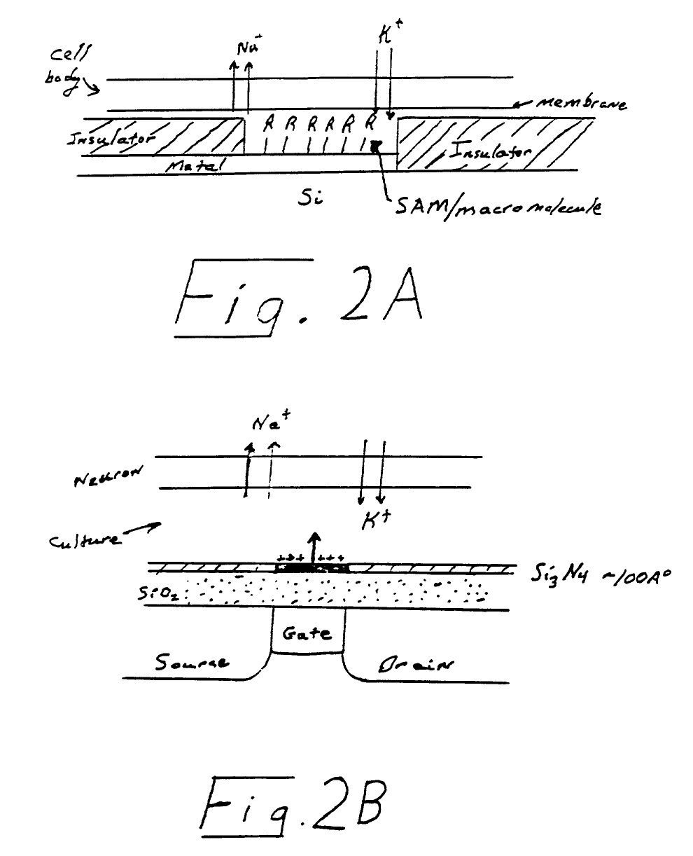

FIG. 2B depicts another aspect of the invention in which an insulating layer and barrier layer are provided between the neuron and a FET.

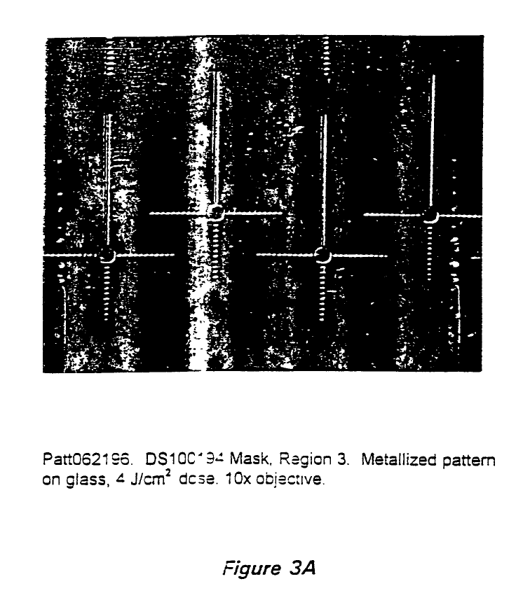

FIGS. 3A and 3b depict metallized patterns on a silicon substrate for imparting a defined polarity to cells provided in a biosensor according to the present invention.

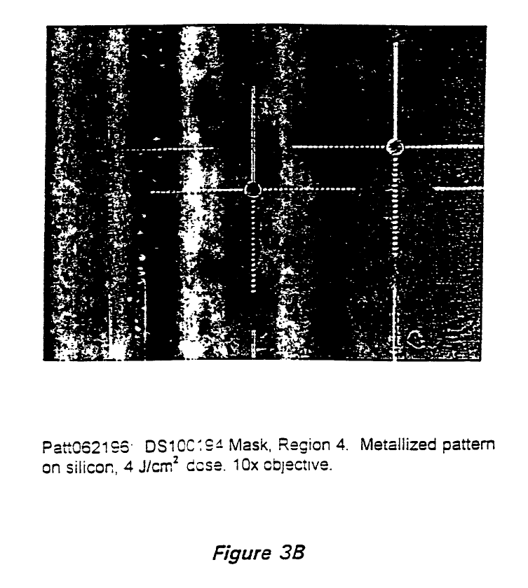

FIGS. 4A and 4B depict photographs of mask features and patterned growth of hippocampal neurons having a so defined polarity according to the principles of the present invention.

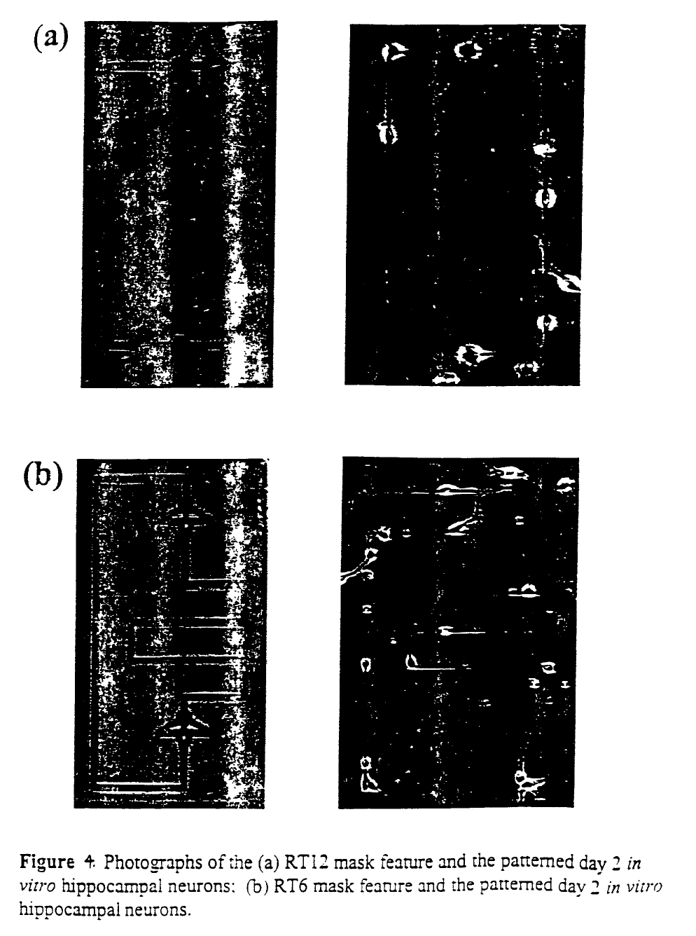

### 4. DETAILED DESCRIPTION OF THE PREFERRED EMBODIMENTS

According to the principles of the present invention, simple and complex circuits are constructed from mammalian cells, particularly mammalian neuronal cells, oriented in a predetermined way with respect to one another and with respect to one or more transducers to create a biosensor. This sensor can act not only as a screen for known compounds but unknowns as well. These function-based sensors can detect toxins or environmental effects ranging from the obvious (cell death) to those that are more subtle (impairment of function). For instance, substances inhibiting, enhancing, or otherwise affecting the electrical signal output from a natural neuronal network can be found by a screening method utilizing a neuronal network or a biosensor incorporating the network. It is possible under the invention to measure a wide range of responses because neurons and the networks they form are exceedingly sensitive to certain changes in their environment. The development of a solid state device to monitor neuronal signals combined with surface patterning enables a novel sensor based on rudimentary cognitive function.

Previous work has been reported on measuring signals from neurons using solid state devices (Eggers et al., 1990; Fromherz et al., 1991; Regher et al., 1989, Stenger et al., 1993). However, in these previous reports, the measurement was of changes in the cell membrane potential due to the flux of ions across the membrane, e.g., with a microelectrode. In contrast, a biosensor of the present invention can sense an axonal wave potential propagating through an electrically active cell. For instance, by monitoring the electrical signals between two neurons in defined circuits and in defined growth media, with geometrically or biologically induced polarity, one can detect changes in cell-cell communication as when an analyte is introduced into the medium. A sensor based on cognitive function to screen the environment to detect toxins or other bioeffecting materials may also be used in concert with sensors targeted for specific known bioeffecting environmental conditions, e.g., toxins.

Crude patterns of rat hippocampal neurons have been made on insulators. The patterns are achieved by creating artificial surfaces using self-assembled monolayers (SAMs) in in vitro cell culture as described by Hickman et al., 1994. These artificially created surfaces are designed to be amenable to photolithography, such that two-dimensional geometric patterns can be created on the surface. A surface modified with a self-assembled monolayer comprised of N-2-aminoethyl-3-aminopropyl-trimethoxysilane (EDA) supported neuronal growth best in serum-free medium rather than in complex, undefined, serum-containing medium. This observation has been extended to other SAM surfaces.

The demonstration that neurons grow best on SAM-modified surfaces in defined medium was a crucial requirement for the use of patterned neurons as the basis of a sensor system because any changes noted in cell-cell communication after the introduction of a waste stream would be due only to that addition. Patterns have also been achieved on insulators establishing that the fabrication of a solid-state sensor is possible.

The present invention thus provides for a biosensor for determining the presence of bioeffecting environmental conditions, toxins and pharmaceutical agents. The biosensor comprises:

(a) a substrate in contact with a culture medium capable of supporting metabolism of one or more cell type;

(b) a cell network composed of at least one viable cell of said cell type, which cell has a predefined axonal/dendritic polarity on said substrate and is capable of producing a signal in response to a bioeffecting substance or condition; and

(c) at least one signal transducer provided proximate said cell network, which is capable of detecting the signal by being operably coupled thereto.

4.1. The Biosensor Substrate

A sensor of the present invention includes a substrate having one or more reactive groups onto which a SAM can form. The only requirement for the surface is that it is capable of having SAMs form thereon and is non-toxic to the cells. Suitable substrates include silica (glass or quartz), Si₃N₄, silicon carbide, metals such as gold and platinum, and the like.

Reactive groups on the substrate may include: —OH, —SH, —-NH₂, —COOH, and the like.

Silica (glass), having —OH reactive groups, is a preferred substrate under the invention as it has a substantially homogeneous structure, is inexpensive, readily available, easily sterilized, non-toxic and stable in culture media. Glass cover slips and polished silicon wafers are examples of this substrate. Also preferred is a silicon nitride substrate such as those wafers commonly used in electronic applications.

4.2. Preparation of the Substrate

A SAM is a modifying layer comprised of organic molecules one molecule thick that can spontaneously form strong interactions or covalent bonds with reactive groups on an exposed surface. The self-assembly of an organosilane on a silica surface is depicted in Stenger et al., 1992, the disclosure of which is incorporated herein by reference.

SAMs can be used to systematically vary the properties of a modified surface since the monomers that comprise SAMs can have a wide range of chemical compositions. A large variety of functional groups can be located on the terminus opposite the attachment point (Ulman, 1991), and a combination of functional groups allows the chemical composition of the resulting monolayer to be manipulated to tune the surface free energy (Stenger et al., 1992). Most importantly, SAMs provide a means to geometrically pattern a surface (Stenger et al., 1992; Hickman et al., 1994) on which cells can be subsequently grown. SAMs thus prove an ideal tool for the design of circuits or surfaces for the study of neuronal interactions and their incorporation into biosensor design.

In the practice of the invention, the substrate is modified by reaction with one or more molecules capable of forming a self-assembled monolayer, in particular for use with a silica, Si₃N₄, or SiC substrate.

The first SAM surface is generally chosen to be one that promotes cell, particularly neuronal, adhesion and growth. The modified surface may then be subject to photolithography using known techniques, followed by re-derivatization with one or more second SAM-forming molecules. These second molecules are generally chosen to be refractory to cell adhesion.

In an alternative approach, the first SAM-forming species may be one that is refractory to cell adhesion. In this case, after photolithography, the surface is re-derivatized with a SAM-forming molecule that promotes cell adhesion and growth.

If additional patterning is desired, additional photolithography steps can be performed followed by the addition of one or more additional SAM-forming molecules.

The patterned surface created on the substrate may be of any predetermined geometry. In general, the pattern includes at least two areas of sufficient size for the adhering of a cell body, preferably a neuronal cell body. These cell-body areas are interconnected via at least one region of adhesion of sufficient width to provide for adhesion of at least one axon.

Several properties of the SAM can be varied. For example, the terminal functional groups, which can be small charged groups such as amines, thiols, carboxylic acids, sulfonic acids, can be used. Large moieties, such a bipyridine rings, can be placed so as to control the hydrophobicity/hydrophilicity of the molecules. The length of the carbon chain to which these different functional groups are attached can also be varied, as it is known that the surface free energy (a measure of the hydrophobicity) can be varied simply by varying the relative length of the anchor carbon chains. These SAMs can be further derivatized by charge-induced condensations, heterobifunctional crosslinkers and simple adsorption.

4.3. Protein Derivatization of the Substrate

In further derivatization, molecules that stimulate the growth and/or adhesion of the cells can be used. For neural cells, proteins such as serum albumin, laminin, tenascin, NCAM, L1, bFGF and GAGs can be used. Of these, serum albumin may play an important role in supporting the differentiation of embryonic cells which may be important in encouraging the attachment to de novo tissue. Albumin also can prevent the non-specific adsorption of other proteins.

Laminin, tenascin, NCAM and L1 cause interactions with the extracellular matrix of various tissues. Laminin encourages neuronal growth and the branching of microglia. NCAM may be able to induce proliferation of neurons. Tenascin has been shown to be repulsive to neurons and glia, and may be useful as a derivatization of cell-repelling regions. L1 has been shown to encourage axonal migration and can therefore be useful as a derivatization of regions supporting axonal growth.

The growth factors bFGF and BDNF are known to be used in the signal transduction pathways leading to cellular differentiation and have been shown to promote neuronal survival in culture.

The GAG polysaccharides represent the other major class of extracellular macromolecules that make up the ECM.

After preparation, the substrate is generally sterilized. This is easily accomplished by soaking the substrate in one or more solvents, for example, ethanol. The substrate is then allowed to air dry, preferably in a sterile environment.

The patterned surface created on the substrate may be of any predetermined geometry. In general, the pattern includes at least one area of sufficient size for the adhering of a cell body (soma), preferably a neuronal cell body. These cell-body or somal areas are interconnected via at least one region of adhesion of sufficient width to provide for adhesion of at least one axon or dendrite. For hippocampal neurons, the somal region is about 20-25 μm in diameter. The somal region comprises a cell-adherent SAM, preferably comprising EDA or DETA, and is that area of the device to which the neuron cell-body attaches.

Connected to the somal region is at least one continuous path of cell-adherent SAM, for example DETA, of sufficient width to provide for the growth and adhesion of an axonal process. For rat hippocampal neurons, for example, the width of the continuous path is thus generally from about 2.5 μm to about 25 μm, preferably from about 2.5 μm to about 10 μm, most preferably about 5 μm.

Further connected with the somal region are one or more discontinuous paths that comprise a cell-adherent SAM interrupted by a plurality of intermittent segments of at least one type of non-cell-adherent SAM. The non-cell-adherent intermittent regions of the discontinuous path are preferably made during the re-functionalization (i.e., re-derivatization) of the molecular template after photolithography. The width of the one or more discontinuous paths is generally that sufficient to provide for attachment of one or more dendrites. For rat hippocampal neurons, the width of the one or more discontinuous paths is from about 2.5 μm to about 25 μm, preferably from about 2.5 μm to about 10 μm, most preferably about 5 μm. The spacing between the intermittent non-cell-adherent regions of the discontinuous path (i.e., the depth of the cell-adherent portion of the discontinuous path) is generally from about 0.25 μm to about 25 μm, preferably about 2.5 μm, while the width of these regions is from about 1.-0 μm to about 10 μm, preferably about 5 μm.

4.4. Geometric Configuration of Cell Network and Electrical Circuit Elements

The accurate spatial placement of a neuronal cell network allows one skilled in the art to apply a wide spectrum of circuit and fabrication technology to the detection of signals transmitted within the network. The formation of cell networks in which the cells have a predetermined axonal/dendritic polarity is the subject of U.S. Ser. No. 08/689,970, filed Aug. 16, 1996, the disclosure of which is incorporated herein by reference.

Preliminary work by Fromherz et al. (1991) demonstrated that field effect transistors (FETs) can detect membrane potential changes from cell bodies. Membrane potentials also occur in the connections between cells. An axon propagates its signal by transferring ions through channels in the axon membrane. Typically, Na⁺ or Ca²⁺ ions are transferred from outside the membrane to inside, followed some time later by the transfer of K⁺ ions from inside to outside. Normally a potential difference exists between the inside and outside of the membrane on the order of tens of mV, with the inside being negatively charged relative to the outside. When the Na⁺ or Ca²⁺ channels open, this potential difference is momentarily absent; some few milliseconds later, the K⁺ channels open and the potential difference is reestablished. These changes in potential are measured by a transducer of sufficient sensitivity in the biosensor of the invention.

In a preferred aspect, the unpatterned side of the substrate is equipped with at least one transducer placed in a predetermined geometry with respect to a predetermined cell network pattern. In general, preferred embodiment has at least two transducers are positioned to detect an axonic wave potential in a communication between the at least two cells. Where there are more than two cells adhered to the substrate, there may be additional transducers to detect the axon wave potentials of more than one cell. Thus, the biosensor substrate under the invention may have a small or large number of cells adhered thereto and a small or large number of transducers connected therewith, with substantially each of the cell connections associated with at least two transducers.

Transducers in the biosensor of the invention include, for example, field effect transistors, microelectrodes, photodiodes, piezoelectric materials, fiber optic devices, conductive polymers, liquid crystals, spectroscopic apertures, and the like. Additionally, there may be more than one type of transducer in the biosensor of the invention. The preferred transducers are field effect transducers (FETs).

The measurement of such a dipole oscillation as it is transmitted down the axon can be performed by the use of multiple pickups. By putting multiple FET pickups on the long axon legs of the neuronal pattern, the movement of the membrane wave potential can be detected and measured. These pickups are the gate electrodes of an insulated gate FET located on the neuronal axon. Changes in the wave potentials are used as an indicator of the presence of one or more bioeffective substances.

The sensor signal in the presence of test sample can also be compared to standard physiological recordings (patch-clamp) by measuring the fields generated by low current. Furthermore, as the patch clamps can stimulate as well as record cell signals, several signature frequencies can be isolated and then be used to monitor “normal” cell-cell communications. The performance of the toxin sensor or drug discovery tool is easily assessed by adding known compounds to the system and recording the changes that occur in cell-cell communication.

The solid state/biological interface is tailored under the invention, such that reproducible patterns can be made having desirable surface properties.

4.5. Cell Preparation

Cells are arranged on the substrate in a predetermined pattern. Any cell type, or combination thereof, may be used according to the principles of the invention, so long as they provide defined intercellular connections.

The cells may be derived from tissue, primary culture, stem cell derived cell line, or tissue culture origin. Cells derived from embryonic tissue are most preferred. In a preferred embodiment, the cell type used in the biosensor network is one or more mammalian neuronal cell types, for example, hippocampal cells.

In a more preferred embodiment, the cells are embryonic hippocampal cells, cortical cells or spinal cord cells, preferably embryonic mammalian hippocampal and/or cortical cells. The most preferred cell-type is embryonic rat hippocampal cells.

If a tissue source is desired, it must first be dissociated by, for example, mechanical homogenization or enzymatic methods. An example of such homogenization is the multiple passing of the tissue sample through a constricted pipette. After dissociation, the cells may be washed in, for example, culture medium such as DMEM containing 10% fetal bovine serum, followed by one or more washes in serum-free medium and resuspension in a predetermined volume of serum-free, defined culture medium.

When prepared from monolayer tissue culture, calcium chelators such as EDTA and EGTA may be used to remove the cells from the tissue culture substrate. Such chelators are preferably in a calcium and magnesium-free, buffer, such as calcium- and magnesium-free phosphate buffered saline. The cells may then be collected by low speed centrifugation, optionally washed one or more times, and subsequently resuspended in a predetermined volume of serum-free, defined culture medium.

In one embodiment of the invention, hippocampal neurons from E18 rat embryos are isolated from timed-pregnant Sprague-Dawley rats (Taconic Farms, Germantown, N.Y.). Hippocampi are excised and enzymatically dissociated with papain into single cell suspensions as described previously (Heuttner & Baughman, 1986; Schaffner & Daniels, 1982). Cells are plated onto the SAM-modified substrate at a density of about 5×10⁴ cells/cm². The modified surfaces had been sterilized in 70% ethanol for 3 minutes prior to cell plating. After plating, the substrate is placed in a culture dish and the cells are grown at about 37° C. in a humidified 5% CO₂/air atmosphere. After sufficient time (e.g., 30 min) for cell attachment and growth, at least two transducers of the biosensor are electrically connected to one or more signal detection or signal storage means. The signal detection means preferably includes an amplifier means. The signal storage means may also be used in conjunction with the biosensor.

4.6. Biosensor Operation

In the practice of one embodiment of the invention, one or more samples to be tested are contacted with the biosensor, with the biosensor preferably being in contact with a predetermined amount of media, preferably serum-free media. The axonic wave potential is then monitored or recorded through use of the at least two transducers.

The signal may be monitored immediately or recorded for later analysis and/or processing. Changes in the axon wave potential relative to that potential prior to the addition of the test sample are indicative that the sample contains one or more bioeffecting materials. Alternatively, the axon wave potential in the presence of the test sample can be compared to the axon wave potential of a “control” biosensor wherein no test sample has been added.

In the case of testing for a known bioeffecting material, the change in axon potential induced by the test sample may be compared to a “control” in which a predetermined quantity of the known bioeffecting material is present.

The data from the at least two transducers may be recorded at any predetermined time after the addition of the test sample to the biosensor culture media. Alternatively, the data may be taken continuously for a predetermined period of time.

Alterations in the axon wave potential indicative of the presence of a bioactive substance in the test sample include, for example, the wave speed, periodicity, intensity, duration, and phase.

Examples of bioeffecting materials whose presence can be detected include: toxins such a channel blockers, pharmacological agents, growth factors, hormones, living organisms, such as bacteria, fungi, protozoa, etc.

When two or more cells are plated on the molecular template, the neuronal circuit is complete whenever the axon of at least one neuron extends toward the dendrites of at least a second neuron at a distance sufficient to allow communication between these at least two neurons. For hippocampal neurons, these distances can vary from about 100 to about 600 μm. Depending on the type of neuron(s) and the type of synapse formed, however, the range in distances can be as small as tens of microns to as large as hundreds of microns, even millimeters. For neuronal circuits comprising more than two neurons, circuits of increasing complexity can be created in which, for example, substantially all of the cells are interconnected and in communication with at least one other cell. Synapses formed may be either excitatory, which cause depolarization of the post-synaptic cell upon stimulation, or inhibitory, which cause hyperpolarization of the post-synaptic cell, depending on the types of neurons interacting.

Neuronal circuits, such as the simple circuit described, are essential to the production of a biosensor of the invention. For example, such a simple circuit can be used to assay a test solution for the presence (or absence) of one or more bioeffecting substances (i.e., in this case, substances that exert a measurable effect on the frequency, intensity and/or phase of the interconnected and communicating cells). Such substances are detected, for example, by recording or monitoring alteration in the character of the axonic wave potential due to the presence of the bioeffecting substance.

Neuronal circuits under the invention are also useful in evaluating the pharmacologic activity of potential drugs, in particular, neuroactive drugs. Changes in neuronal communication, for example, can be detected by bioelectronic sensors of the invention. Such changes may include increases or decreases in the axonic wave characteristics such as propagation speed, intensity, duration, frequency, phase, etc., as well as changes in one or more characteristic of one or more cells in the biosensor, the characteristics of the overall biosensor, and/or changes in the characteristics in one or more biosensors in a more complex circuit.

When desired, a solution containing a known pharmacologic agent can be used as a control for comparison to the activity of a test substance. Accordingly, such changes in neuronal communication, in turn, allow the monitoring of substances having pharmacologic activity, whether detrimental, beneficial, or innocuous. Such changes in cell communication are monitored, correlated and/or compared with changes produced by known pharmacological agents. In this manner, new pharmacological agents can be identified, which have similar or different degrees of potency relative to that of a known agent. Similarly, the presence of any pharmacologic agent, known or unknown, can be detected.

5. Experimental

The PL and organosilane films were deposited on glass cover slips (22 mm×22 mm, 0 thickness) purchased from Thomas Scientific, Swedesboro, N.J. or, alternatively, on 50 mm diameter <100> n-type polished silicon wafers obtained from Virginia Semiconductor, Fredricksburg, Va. Glass surfaces are first cleaned using 1:1 Hcl:methanol followed by a concentrated H₂SO₄ soak for 30 minutes followed by a water rinse. PL films were deposited by exposing clean surfaces to 20 μg/ml PL dissolved in sterile, tissue culture grade water for 1 hour at room temperature. The EDA and DETA films were formed by reaction of cleaned surfaces with 0.001-1% (v/v) mixtures of the organosilane in 94% (v/v) 1 Mm acetic acid in anhydrous methanol and 5% water for 15 minutes (Hickman et al., 1994).

The surface properties were determined using XPS, optical ellipsometry, and wettability measurements. XPS and ellipsometry indicated differences in film thicknesses as well as oxidation states of the amine groups.

Cells were cultured in a modified serum-free N3 media, mechanically dissociated, and plated at 10⁴-10⁶ cells/cm². The efficacy of SAMs were then related to the physical and chemical properties of the surfaces.

5.1. Surface Effects on Neurite Growth

Polylysine (PL) was used as a standard to which five SAMs were compared. It was seen that, after 72 hours, the ability of surfaces to promote the adhesion and rapid outgrowth of neurites was sensitive to the availability of protonated amine groups, and had no apparent relationship to the surface density of unprotonated amines. The exceptions were PEDA and BIPY, suggesting that aromatic rings present in PEDA and BIPY are detrimental to neuron survival.

A caprolactam (CL) which, unlike EDA, PEDA, and PL, is not protonated at physiological pH was also investigated. After 24 hours, the neuron growth was excellent and even more extensive than it was on PL. However, after 72 hours the neurons began to deteriorate. On the basis of these observations, it was concluded that a positively charged surface is not essential for initial neuron survival and neurite outgrowth; however, increasing positive charge appeared to be beneficial for long term survivability. It is thus apparent that the molecules which comprise the monolayer are important to the substratum interactions, and that the functional groups play a major role in neuron survival and neurite outgrowth, at least in certain cases.

Another important aspect of a biosensor of the invention is SAM stability under physiological conditions. Stability studies of certain SAMs in phosphate buffered saline (PBS) at 37° C. showed that some SAMs are unaffected by the treatment after 24 hours while others have degraded or changed state. However, several classes of cells including neurons (Stenger et al., 1992; Hickman et al., 1994), neuroblastoma (Georger et al., 1992), and endothelial cells (Spargo et al., 1994) have all maintained adherence to geometrically defined monolayer patterns for up to two weeks in culture (the longest time point tested). This result shows that the effects of the monolayers are sustainable for at least two weeks, whether or not the monolayers are still present.

Other SAMs such as fluorinated alkyls, and silanes containing functional groups such as thiols, sulfonic acids, and other amines influence the growth of cells in culture. Other important factors in in vitro cell culture include the cell type, the growth medium, and cell dissociation method. Using the methods described herein these variations of the invention can be studied and optimized by a person skilled in the art using routine experimentation.

In one embodiment of the invention, the following silanes were used to prepare SAMs:

1. EDA N-(2-aminoethyl)-3-aminopropyl-trimethoxysilane

2. DETA trimethoxysilylpropyldiethylenetriamine

3. MTS 3-mercaptopropyltrimethoxysilane

4. MTSox oxidized MTS (Bhatia et al., 1992)

5. TP triphenylchlorosilane

6. OTS octadecyltrimethoxysilane

7. PEDA (aminoethylaminomethyl)phenethyltrimethoxysilane

8.13F (tridecafluoro-1,1,2,2-tetrahydrooctyl)-1-dimethylchlorosilane

Silanes #1-4 have been demonstrated to support adhesion and growth of embryonic neurons in vitro while silanes #5-8 do not (Kleinfeld et al., 1988; Stenger et al., 1993; Hickman et al., 1994). SAMs are prepared as described above. Coverslips modified with poly-D-lysine (PL) serve as standard or “control” culture substrata.

Two surface analytical techniques are applied to the characterization of SAM-modified coverslips prior to their use in culture experiments: contact angle measurements and X-ray Photoelectron Spectroscopy (XPS). Contact angle measurements quantify wettability and serve as indications of the reproducibility of the SAM preparation methods (Bain & Whitesides, 1988). A representative coverslip from each batch is examined. Contact angles are measured by application of static, sessile drops (5-30 ul) of deionized water to substrate surfaces, and are made visually on both sides of the drops using a Rame-Hart type goniometer. XPS is used to demonstrate formation of the SAM and to measure the thickness of the monolayer. A representative coverslip is reserved from each batch for XPS, but spectra need only be obtained for establishing surface composition

XPS is a technique for the elemental analysis and characterization of surfaces (Briggs & Seah, 1992). XPS measurements are obtained on a FISONS 220i spectrometer with imaging capability to 2 μm resolution. All spectra are referenced to the Si 2p3/2 peak of SiO₂ or Si₃N₄. Any surface charging can be neutralized with a 2.7 eV electron beam through a thin metal mesh screen.

XPS element survey scans illustrate the differences in the amounts of certain elements on EDA surfaces after culture in MEM-S, HS/EDA in MEM-S, and EDA in MEM-N3. The relative amount or material on each surface was deduced by XPS from the magnitude or the Si peak resulting from the underlying silica substrate, since the larger the Si peak, the less material adsorbed to and attenuated the surface signal. In serum-containing medium, the amount of material deposited on the EDA/HS surface was less than on EDA alone, demonstrating that, at least in this case, the HS may prevent proteins from adsorbing to the surface.

It has been found that the relative amounts of C, O, and N are different when cells are cultured in serum-free media on EDA. Thus, in the absence of serum, the substrate may be able to play a dominant role in defining the nature of the cell-surface interaction.

5.2. Media Effects on Neurite Growth

One problem faced in the quantitative study of cell-surface interactions has been that most neuronal preparations on PL use serum-containing medium, which is compositionally ill-defined. To determine the differences between culture in serum and serum-free growth media on SAM-modified surfaces, the different conditions reported by several labs to sustain cell growth were investigated. (Banker & Goslin, 1991; Schaffner et al., 1987; Banker & Cowan, 1977; Brewer & Cotman, 1989).

Glass coverslips were coated with either PL or EDA. Another set of EDA-modified substrates was incubated overnight at 37° C. with heparin sulfate (HS) before cells were plated. Hippocampi from 18 day rat embryos were mechanically dissociated or enzymatically dissociated with papain into single cell suspensions (Heuttner & Baughman, 1986). Cells were plated onto coverslips and cultures were maintained in minimum essential medium (MEM) with 5% fetal calf serum and 5% horse serum (MEM-S) or in serum-free medium (MEM-N3) according to Romijn (Romijn et al., 1984). All cultures were maintained in a humidified atmosphere of 9.5% CO₂/air at 36° C. At various time points, cells were examined and photographed. Each permutation of dissociation method/medium/surface was run in duplicate. In all cases, the overall appearance of the neurons from duplicate cultures was the same.

The experiment was performed in parallel with papain dissociated cells (data not shown). In MEMS, PL (top left) supported hippocampal neuron survival with extensive neurite outgrowth, while EDA and EDA/HS modified surfaces did not (middle and bottom left), regardless of dissociation method. In contrast, in MEM-N3, PL (top right) did not support survival as well as EDA or EDA/HS (middle and bottom right). These findings demonstrate that neurite survival in serum-containing (MEM-S) and serum-free medium (MEM-N3) were dramatically different, with PL performing well as a substrate in MEM-S, but EDA and EDA/HS working well in MEM-N3. These results further demonstrate that artificial surfaces created using SAMs can support hippocampal neurons in culture in serum-free media. Such a growth system permits an environmental biosensor wherein the only unknown is the introduction of a waste stream or toxin.

5.3 Characterization of Patterned Neurons on SAM-Modified Surfaces

To determine appropriate SAM surfaces for supporting the growth and survival of E18 hippocampal neurons, the following experiments were performed. Four different silanes, EDA, DETA, MTS, and MTSox, were assembled on silica (glass) surfaces essentially as described. Silica was again used as the substrate to provide for a unique signal for XPS analysis. As above, the neurons were cultured in serum-free media. Initially the neurons were cultured on homogeneous surfaces, i.e. prior to patterning. Taken together, these data allow quantitative evaluation of the effect of each SAM-modified surface on cell growth and development. A poly-lysine (PL) standard was used for each experiment in order to assess the general health of the original cell suspension in the event that there was an immediate and considerable loss of cells in vitro.

Each SAM-modified surface was characterized before each experiment by contact angle, and XPS if appropriate. XPS is a technique for the elemental analysis and characterization of surfaces that allows determination of whether quantities such as the starting density of functional groups or their oxidation states can be correlated to cell growth or phenotype. Characterizing surface hydrophobicity or hydrophilicity is used to determine whether the relative hydrophobicity or functional group accessibility can be correlated to cell adhesion, growth, or phenotype.

E18 hippocampal neurons were next cultured on patterned surfaces. A two-cell circuit was prepared such that the axon of the top neuron is directed to impinge on the dendrites of the bottom neuron. It is now possible to induce polarity in these neurons using geometric surface clues. Phase microscopy can be used to assess which SAM combinations result in specifically placed cell bodies and appropriate neurite extension along the desired pathways.

Since the neurites follow the designated pattern paths, the electrical properties of hippocampal neurons in two-cell circuits were monitored by electrophysiological recordings, e.g., patch clamp recordings, from pairs of neurons. These recordings were then compared to the recordings made on cells that were not patterned.

Electrophysiological recordings from cells patterned on SAM-modified silicon nitride (Si₃N₄) surfaces were also be obtained. It was found that neurons cultured on SAM-modified Si₃N₄ have similar morphological properties to those plated on SAM-modified glass. In these experiments, silicon nitride surfaces were first activated by exposure to an oxygen plasma.

5.4 Electronics Design and Fabrication

In the FET detection mechanism, the gate electrode contact is covered by a layer of SiO₂ and then a layer of Si₃N₄. The Si₃N₄ layer prevents diffusion of the ions from the growth medium into the electrical circuitry, and thereby serves to lower the background levels and increase the sensitivity of the detection of neuronal activity levels. The volume between the outermost layer of Si₃N₄ and the axon membrane was initially filled with some concentration of, as an example, Na⁺ ions. During the initial phase of the propagation of the axon signal, the Na⁺ channels open and Na⁺ ions are transported across the membrane. At frequencies significantly less than 10 MHz, the membrane appears as a high impedance, and this flowing Na⁺ charge is “lost” from the volume between the membrane and the gate pickup. The rapid loss of charge from this volume induces a charge polarization on the insulators over the gate pickup, producing a voltage impulse to the gate electrode. Some time later, the K⁺ channels open and K⁺ ions flow in the reverse direction from the other side of the membrane, and a similar but opposite sign impulse is imparted to the gage electrode.

Previous measurements have shown that the potential swings at the cell body are on the order of ˜20 mV and monopolar (Fromherz et al., 1991). By contrast, the axonal process signals are thought to be bipolar and much smaller in magnitude. The peak axonal current oscillation is estimated to be on the order of 10-12 A, with a minimum timescale of 1-0.01 msec. This corresponds to an oscillating charge of 10.00 fC. The typical gate-source/drain capacitance of a microfabricated FET is −1 pF, and a voltage signal of 1 mV is straightforwardly managed with subsequent amplification. The minimum charge that is therefore required to be induced on the buried gate electrode is ˜0.001 fC. While this is less than the charge oscillating in the axon, careful placement of the gate electrode and management of the additional stray capacitances resulting from the interaction of other detection components with the surrounding electrolyte solution provides accurate measurements.

The precise signals that are coupled to the gate electrode and out to the detection systems are dependent on a number of parameters that are open to design, modelling, and optimization, as recognized by those skilled in the art. Another important output to be considered is the frequency dependence of the FET measurement system itself. Where necessary, signals can be post-processed to remove this dependency.

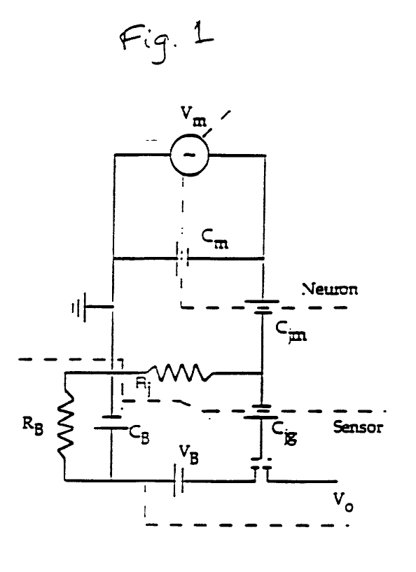

An exemplary circuit model of a biosensor according to the present invention is shown in FIG. 1. Electronic circuitry for use with the present invention is essentially that shown by Fromherz et al. (1991), with the exception that the FET is explicitly shown, as are possible feedback paths of the bias voltage. As shown, the membrane voltage, Vm, is taken to be an idealized AC source in parallel with the membrane capacitance, Cm. Any voltage appearing across the membrane appears across this capacitance in parallel with the junction-membrane capacitance Cjm. The signal then has the option of leaking to the outside world through the junction resistance Rj, or coupling to the FET pickup through the junction-gate capacitance Cjg.

As indicated above, the pickup FET is explicitly shown, with the drain voltage signal taken out as the preamplified output signal. The circuit model also explicitly shows the possible feedback of the FET signal through a coupling capacitance and leakage resistance to the surrounding solution, these are shown as CB and RB respectively.

There are several elements of a circuit of the invention that are amenable to electronic circuit design, electronic package design, and overall sensor design optimization. The parameters of the FET itself are open to significant design tradeoffs of sensitivity, signal to noise ratio, input impedance, and frequency roll-off. As known to those skilled in the art, the package design takes into account optimizations of the feedback parameters CB and RB, as well as selection of where to take the “ground” line for the output signal VO.

5.5. Characterization of Two-Cell Circuits

Circuits may be characterized using dual-patch clamp recordings in the whole-cell mode or inter-cellularly. Brief electrical stimuli (on the order of milliseconds) of varying intensity are delivered in a systematic manner to one of the neurons to evoke signals in the adjacent neuron.

From the position of the circuit in the 10×10 grid, one identifies the pair of cells that is subject to recording, and then revisits the pair to stain for specific molecules of interest. For example, one can use standard immunostaining techniques to identify synapse-specific proteins, as well as stains for specific neuronal phenotypes.

5.6. Microfabricated Circuits

Micro-fabricated circuits were rigorously tested to characterize their adherence to design specifications. Test leads are placed on the FET input in an approximation of the desired cell geometry. These leads are driven with signals from 0.1 to 10⁶ Hz and peak values of 1-100 mV. The resulting circuit output signal was measured for the complete range of FET bias voltages.

The final measurement series is performed on fully assembled sensors, including patterned neurons. The measurements in this configuration focused on single-sensor measurements of the cell, axon, and dendrite signals. These were followed by simultaneous measurement of axonal signals at several positions along the length of a single axon. In particular, these measurements characterize the nature of the signal as it propagates along the axon in terms of pulse shape, frequency content, phase delay, and the absence or presence of fast-timescale signal precursors. In the practice of the invention, measurement of changes in the axonal pulse is indicative of one or more bioeffecting substance(s), such as a toxin(s) in the medium, and thus provides for a sensitive biosensor regardless of whether the identity of the bioactive substance is known. Indeed, using a biosensor of the present invention in conjunction with standard analytical techniques, many previously unknown bioeffecting molecules can be isolated and identified.

5.7. Further Examples:

(1) A biosensor having:

(a) a substrate having at least one signal transducer in a predetermined geometry and capable of detecting an electrical signal;

(b) a defined culture medium selected to support the metabolism of one or more cell-type;

(c) at least one barrier layer interposed between said transducers and said culture medium, said barrier layer preventing direct contact between said culture medium and said transducers, wherein said barrier layer comprises an insulator; and

(d) a reproducible cell network comprising at least two viable cells of said one or more cell type established over said barrier layer in a predetermined geometry and polarity, wherein at least one of said one or more cell type is capable of producing said signal.

(2) A biosensor where the signal transducers are field effect transistors, microelectrodes, or potentiometers.

(3) A biosensor having a metal microelectrode covered with a SAM that forms a high impedance seal with the cell.

(4) A biosensor having a layer of silicon nitride, silicon carbide, or other insulating material, over a layer of silicon dioxide.

(5) A biosensor having a self-assembled monolayer covering a layer of insulating material, which self-assembled monolayer is patterned.

(6) A biosensor composed of at least one electrically excitable cell of one or more viable neuronal cells, neuronal stem cells, or neuronal cell lines.

(7) A biosensor where the detected signal is a cell membrane wave potential.

(8) A biosensor having a neuronal cells that are cells of hippocampal, cortical or spinal cord origin.

(9) A biosensor having (a) to (d) above and also having a means for measuring a signal produced by the two transducers.

(10) A biosensor having a least two viable mammalian cells.

(11) A biological sensor that incorporates the neuroelectric logic device circuitry described in U.S. provisional patent application Serial No. ______, filed May 6, 1997, which is incorporated by reference. Such a biosensor includes:

(a) a cell network of at least two viable cells of one or more cell types including at least one neuron capable of producing a detectable axonal or dendritic signal;

(b) a defined culture medium selected to support the metabolism of the at least two viable cells;

(c) a substrate having at least two signal transducers arranged in a predetermined geometry and capable of detecting the signal, with one or both of the transducers optionally capable of stimulating/establishing a signal in at least one of the cells; and

(d) an insulator or barrier layer on the substrate that prevents direct contact between the culture medium and the transducers; and, where the cell network is established on the insulator or barrier layer in a predetermined geometry and polarity such that at least one of the transducers is capable of detecting a signal produced by the at least one neuron.

(12) A biosensor where the axonal signal is an axonal wave potential.

(13) A method for assaying a bioeffecting substance including the steps of:

(a) providing a biosensor having components (a) to (d) above, where one of the at least two cells produces a detectable response to at least one bioeffecting substance, and a test sample;

(b) contacting the test sample with the biological sensor;

(c) measuring a response produced by one of the at least two cells; and

(d) correlating the response to the presence or absence of a bioeffecting substance in the test sample.

(14) A method for assaying a bioeffecting substance where the bioeffecting substance is a toxic substance.

(15) A method for assaying a bioeffecting substance where the bioeffecting substance is a pharmacological agent.

(16) A method for assaying toxic substances where the identity of the toxic substance is known.

(17) A method for assaying toxic substances where the identity of the toxic substance is unknown.

(18) A process for making a biosensor including the steps:

(a) providing a planar substrate member having at least two sides, and to which one or more predetermined cell types do not substantially adhere and where at least a first side has functional groups;

(b) reacting a composition comprising a substituent to which the cell types are capable of adhering with said functional groups;

(c) removing the substituent to which said cell types are capable of adhering from predetermined areas on the support to provide for continuous paths on the substrate to which the cell types adhere and discontinuous paths to which the cell types do not substantially adhere;

(d) affixing at least two signal transducers to or in the substrate member at predetermined locations;

(e) contacting the first side with a defined culture medium capable of supporting the metabolism of the one or more cell types;

(f) seeding the first side with the one or more cell types in a predetermined pattern; and

(g) using geometric clues to establish polarity of neuron networks.

(19) A process for making a biosensor where at least the first side of the planar substrate has functional groups in a self-assembled monolayer (SAM).

(20) A process for making a biosensor haveing steps (a) to (g) above and connecting the at least two transducers to a signal amplifier.

(21) A process for making a biosensor where the cell types are neuronal cells.

(22) A biosensor for the detection of a substance having biological activity including (a) a transducer functionally coupled to a substrate, and (b) at least two cells supported by the substrate and predisposed to axonal/dendritic polarity, where the transducer is sensitive to electrical signals emanating from the one or more cells, and wherein the substrate supports the one or more cells and has a patterned surface, the pattern including:

(i) zones for the adherence of the cells,

(ii) a solid line extending from the zone in a desired direction, having a length and width effective to encourage the preferential growth of an axon from the cell along the desired direction, and

(iii) one or more broken lines extending from the zone in any direction except the desired direction, each broken line having a length and a width, and in which the breaks in each broken line are of dimensions and frequency, effective to discourage the preferential growth of an axon in any direction except the desired direction,

the zone, solid and broken lines further characterized as being a molecular layer of at least one type of cell-adherent self-assembled monolayer (SAM) and the breaks in each broken line further characterized as being a molecular layer of at least one type of non-cell-adherent SAM.

(23) A biosensor in which the one or more cells are neural cells. A method of screening a substance for potential biological activity comprising contacting a substance suspected of having biological activity with the biosensor sensor having (a) a transducer functionally coupled to a substrate, and (b) at least two cells supported by the substrate and predisposed to axonal/dendritic polarity, where the transducer is sensitive to electrical signals emanating from the one or more cells, and wherein the substrate supports the one or more cells and has a patterned surface, the pattern including:

(i) zones for the adherence of the cells,

(ii) a solid line extending from the zone in a desired direction, having a length and width effective to encourage the preferential growth of an axon from the cell along the desired direction, and

(iii) one or more broken lines extending from the zone in any direction except the desired direction, each broken line having a length and a width, and in which the breaks in each broken line are of dimensions and frequency, effective to discourage the preferential growth of an axon in any direction except the desired direction,

the zone, solid and broken lines further characterized as being a molecular layer of at least one type of cell-adherent self-assembled monolayer (SAM) and the breaks in each broken line further characterized as being a molecular layer of at least one type of non-cell-adherent SAM.

(24) A biosensor in which the one or more cells are neural cells.

(25) A method having steps (a) to (g) above in which the substance exhibits neurologic activity.

(26) A method having steps (a) to (g) above in which the substance inhibits electrical signals.

(27) A method having steps (a) to (g) above in which the substance reduces the intensity of the electrical signals.

(28) A method having steps (a) to (g) above in which the substance alters the intensity, frequency, speed and/or phase of the electric signals.

Although the present invention has been described with reference to particular embodiments described herein, the invention is not limited thereby. Various modifications and improvements, which fall within the scope of the appended claims, will become apparent to those skilled in the art based upon the foregoing description.

The pertinent disclosures of each of the patents, patent applications, and publications cited herein are incorporated herein by reference.

### 9. REFERENCES

Adinolfi, M., and Haddad, S. A (1977). Levels of plasma proteins in human and rat fetal CSF and the development of the blood-CSF barrier. Neuropadiatrie 8, 345-353.

Aizawa, M. (1991). Immunosensors. In Blum, L. J., & Coulet, P. R. (Eds.), Biosensor Principles and Applications pp.249-266, (Marcel Dekker, Inc: NY).

Anderson, J. M, Bonfield, T. L. & Ziats, N. P. (1990). Protein adsorption and cellular adhesion and activation on biomedical polymers. Int. J. Artificial Organs 13, 375-382.

Bain, C. D. and Whitesides, C. M. (1988). Correlations between wettability and structure in monolayers of alkanethiols adsorbed on gold. J. Am. Chem. Soc. 110, 3663-3666.

Banker, G. A & Cowan, W. M. (1977). Rat hippocampal neuron dispersed in cell culture. Brain Res. 126, 397-425.

Banker, G. and Goslin, K. (1991). Culturing Nerve Cells (MIT Press: Cambridge).

Barde, Y. A. (1989). Tropic factors and neuronal survival. Neuron 2, 1525-1534.

Baszkin, A., & Boissonnade, M. M. (1993). Competitive adsorption of albumin against collagen at solution-air and solution-polyethylene interfaces. J. Biomed. Mat. Res. 27, 145-152

Bhatia, S. K., Shriver-Lake, L. C., Prior, K. J., Georger, J. H. Calvert, J. M., Bredehorst, R., & Ligier, F. S. (1989). Anal. Biochem. 178, 408-413.

Bhatia, S. K., Hickman, J. J. and Ligler, F. S. (1992). New approach to producing patterned biomolecular assemblies. J. Am. Chem. Soc. 114, 4432-4443.

Blum, L. J., & Coulet, P. R., eds. (1991). Biosensor Principles and Applications (Marcel Dekker, Inc. New York, N.Y.).

Bonfield, T. L., Colton, E. & Anderson, J. M. (1991). Protein adsorption of biomedical polymers influences activated monocytes to produce fibroblast stimulating factors. J. Biomed. Mat. Res. 26, 457-465.

Boulton, A. A. G. B. Baker, G. B., and Walz, W., Eds. (1992) Practical Cell Culture Techniques (Humana Press: Totowa, N.J.).

Brash, J. L. (1991). Role of Plasma Protein Adsorption in the Response of Blood to Foreign Surfaces. In C. P. Sharma & M. Szycher (Eds.), Blood Compatible Materials and Devices pp. 3-24. (Technomic Publishing Co. Inc Lancaster, Pa.).

Brewer, G. J. & Cotman, C. W. (1989). Survival and growth of hippocampal neurons in defined medium at low density: advantages of a sandwich technique or low oxygen. Brain Res. 494, 65-74.

Brigs, M. P. and Seah, M. P. (1992). Practical Surface Analysis by Auger and X-ray Photoelectron Spectroscopy 2nd Ed. (John Wiley & Sons: New York).

Decher, C., Schmitt, J. (1993). Fine tuning of the film thickness of ultrathin multilayer films composed of consecutively alternating layers of anionic and cationic polyelectrolyes. Accepted for publication in Coll. Poly. Sci.

Dziegielewska, K. M., Evans, C. A. N., Lai, P. C. W., Lorscheider, F. L., Malinowska, D. H., Mollgard, K., and Saunders, N. R. (1981). Proteins in cerebrospinal fluid and plasma of fetal rats during development. Devel. Biol. 83, 193-200.

Edelman, P. G., and Wang, J. (1991) Biosensors and chemical sensors, ACS Symposium Series 437.

Eggers, M. D., Astolfi, D. K., Liu, S., Zeuli, H. E., Doeleman, S. S., McKay, R., Khuon, T. S., and Ehrlich, D. I. (1990). Electronically wired petri dish: A microfabricated interface to the biological neuronal network J. Vac. Sci. Technol B 8(6), 1392-1398.

Engel, J. (1992). Laminins and Other Strange Proteins. Biochemistry 31, 10643-10651.

Fraaije, J. G., Norde, W., & Lyklema, J. (1991). Interfacial thermodynamics of protein adsorption and ion co-adsorption. III. Electrochemistry of bovine serum albumin adsorption on silver iodide. Biophys. Chem. 41, 263-276.

Fromherz, P., Offenhausser, A., Vetter, T., & Weis, J. (1991). A neuron-silicon junction: A Retzius cell of the leech on an insulated-gate filed-effect transistor. Science 252, 1290-1293.

Georger, Jr., J. H. Stenger, D. A. Rudolph, A. S. Hickman, J. J., Dulcey, C. S. and Fare, T. L. (1992). Coplanar patterns of self-assembled monolayers for selective cell adhesion and outgrowth. Thin Solid Films 210/211, 716-719.

Goodman, S. L. Cooper, S. L., & Albrecht, R. M. (1991). The effects of substrate-adsorbed albumin on platelet spreading. J. Biomater. Sci. Polymer Edn. 2, 147-159.

Heuttner, J. B. & Baughman, R. W. (1986). Primary cultures of identified neurons from the visual cortex of postnatal rats. J. Neurosci. 6, 3044-3030.

Hickman, J. J., Bhatia, S. K., Pike, C., Cotman, C. W., Shoen, P. & Stenger, D. A. (1994). Rational pattern design for in vitro cellular networks using surface photochemistry. J. Vac. Sci. Tech. A.

Hubbell, J. A., Massia, S. P., Desai, N. P., and Drumheller, P. D. (1991). Endothelial cell-selective materials for tissue engineering in the vascular graft via a new receptor. Bio/Technol. 9, 568-572.

Hughes, R. C., Ricco, A. J., Butler, M. A., and Martin, S. J. (1991). Chemical microsensors, Science 254, 74-80.

Hynes, R. O., & Lander, A. D. (1997). Contact and adhesive specificities in the associations, migrations, and targeting of cells and axons. Cell 68, 303-322.

Kleinfeld, D., Kahler, K. H. and Hockberger, P. E. (1988). Controlled outgrowth of dissociated neurons on patterned substrates. J. Neurosci. 8, 4098-4120.

Lee, S. H. and Ruckenstein, E. (1988). Adsorption of proteins onto polymeric surfaces hvdrophiiicites—a case study with bovine serum albumin. J. Colloid Interf. 125, 365-379.

Leech, D. and Rechnitz, G. A. (1993). Biomagnetic neurosensors, Anal. Chem. 65, 3262-3266.

Leonard, E. F., & Vroman, L. (1991). Is the Vroman effect of importance in the interaction of blood with artificial materials? J. Biomater. Sci. Polymer Edn. 3, 95-107.

Letourneau, P. C., Condic, M. L. and Snow, D. M. (1992). Extracellular matrix and neurite outgrowth. Current Opinion in Genetics and Development 2, 625-634.

Ligler, F. S., Calvert, J. M., Georger, J. H., Shriver-Lake, L. C., Bhatia, S. K, Bredehorst, R. U.S. Pat. No. 5,077,210 (1991).

Massia, S. P., and Hubbell, J. A. (1991). An RGD spacing of 440 nm is sufficient for integrin αvβ3-mediated fibroblast spreading and 140 nm for focal contact and stress fiber formation. J. Cell Biol. 114, 1089-1100.

Nagai, Y., & Tsuji, S. (1988). Cell biological significance of gangliosides in neuronal differennation and development. Critique and proposals. In Ledeen, R. W., Hogan, E. L., Tettamanti, G. Yates, A. J., & Yu, R. K. (Eds.), New Trends in Ganglioside Research. pp. 1329-350 (Liviana Press: Padova).

Nisonoff, A. (1984). Introduction to Molecular immunology (2nd ed.). (Sinauer Associates, Inc: Sunderland, Mass.).

Parce, J. W., Owicki, J. C. Kercso, K. M., Sigal, G. B., Wada, H. C., Muir, V. C., Bousse, L. J., Ross, K. L., Sikic, B. I., and McConnell, H. M. (1989). Detection of cell-affecting agents with a silicon biosensor

Peterson, C. P. (1977). A simplification of the protein assay method of Lowry et al., which is more generally applicable. Anal. Biochem. 83, 346-356.

Pusineri, C. Cazenave, J. -P. (1986). Adsorption at interfaces. In J. -P. Cazenave, J. A. Davies, M. D. Kazatchkine, & W. C. van Aken (Eds.). Blood-Surface Interactions pp. 45-59 (Elsevier Amsterdam).

Regehr, W. G., Pine, J., Cohan, C. S., Mischke, M. D., & Tank, D. W. (1989). Sealing cultured invertebrate neurons to embedded dish electrodes facilitates long-term stimulation and recording, J. Neurosci. Meth. 30, 91-106.

Romijn, H. J., van Huizen, F. & Wolters, P. S. (1984). Towards an improved serum-free, chemically defined medium for long-term culturing of cerebral cortex tissue. Neurosci. Biobehav. 8, 301-334.

Sambrook, J., Fritsch, E. F. and Maniatis, T. (1989). Molecular cloning—a laboratory manual (2nd Ed.) p. 1857 (Cold Spring Harbor Laboratory Press: New York).

Schaffner, A. E. & Daniels, M. P. (1982). Conditioned medium from cultures of embryonic neurons contains a high molecular weight factor which induces acetylcholine receptor aggregation on cultured myotubes. J. Neurosci. 2, 623-632.

Schaffner, A. E., St. John, P. A., and Barker, J. L. (1987). Fluorescence-activated cell sorting of embryonic mouse and rat motoneurons and their long-term survival in vitro. J. Neurosci. 7, 3088-3104.

Solsky, R. L. (1990). Ion-selective electrodes, Anal. Chem. 62, 21R-33R.

Spargo, B. J., Testoff, M. A., Neilsen, T. B., Stenger D. A., Hickman, J. J., & Rudolph, A. S. (1994). Adhesion, spreading, and differentiation of endothelial cells on self-assembled amino- and perfluoro-alkylsilane monolayers. Proc. Natl. Acad. Sci. USA, in press.

Stenger, D. A. Georger, J. H., Dulcey, C. S., Hickman, J. J., Rudolph, A. S., Nielsen, T. B., McCort, S. M. and Calvert, J. M. (1992). Coplanar molecular assemblies of amino- and perfluorinated alkylsilanes: Characterization and geometric definition of mammalian cell adhesion and growth. J. Am. Chem. Soc. 114, 8435-8442.

Stenger, D. A., Pike, C., Hickman, J. J. & Cotman, C. W. (1993). Surface determinants of neuronal survival and growth on self-assembled monolayers in culture. Brain Res. 630, 136-147.

Thoenen, H. (1991). The changing scene of neurotrophic factors. TINS 14, 165-170.

Ulman, A. (1991). introduction to Ultrathin Organic Films (Academic Press. Inc.: San Diego).

van Loosdrecht, M. C. M., Lyklema, J., Norde, W., & Zehnder, A. J. B. (1990). Influence of interfaces on microbial activity. Microbiol Rev. 54, 75-87.

Vroman, L. Adams, A. L., Klings, M., Fischer, G. C., Munoz, P. C. and Solensky, R. P. (1977). Reactions of formed elements of blood with plasma proteins at interfaces. Annals N.Y. Acad. Sci. 283, 65-76.

Ziats, N. P., Miller, K. M., & Anderson, J. M. (1988). In vitro and in vivo interactions of cells with biomaterials. Biomaterials 9, 5-13.

What is claimed is:

1. A biosensor comprising:
(a) a substrate in contact with a culture medium capable supporting metabolism of at least one electrically excitable cell;
(b) a cell network composed of at least one of said electrically excitable cells, which cell has a predefined polarity on said substrate and is capable of producing a signal in response to a bioeffecting substance; and
(c) at least one signal transducer operably coupled to said cell network, which transducer is capable of detecting said signal produced in said cell network.

2. The biosensor of claim 1, wherein the signal produced by said cell network is an action potential, axonal wave potential, or dendritic wave potential.

3. The biosensor of claim 1, wherein the cell is a spinal cord cell, hippocampal cell, CNS excitatory cell line, or a cell line derived from stem cells.

4. The biosensor of claim 1, wherein said defined polarity is defined by a pattern of a self-assembled monolayer or biological macromolecule present on the surface of said substrate.

5. The biosensor of claim 1, wherein the transducer is a field effect transistor or a microelectrode.

6. The biosensor of claim 1, further comprising an insulating and/or barrier layer interposed between said at least one transducer and said culture medium, which insulating and/or barrier layer prevents direct contact between the culture medium and the transducer.

7. The biosensor of claim 6, wherein said insulating and/or barrier layer is selected from the group consisting of silica, silicon, germanium, gallium, arsenide, epoxy resin, polystyrene, polysulfone, aluminum, platinum, alumina, silicone, fluoropolymers, polyesters, acrylic copolymers, polyglactin, and polylactates.

8. The biosensor of claim 7, wherein the insulating and/or barrier layer comprises silica, silicon nitride, or silicon carbide.

9. The biosensor of claim 1, wherein said substrate has a patterned surface with at least one region thereon having an exposed surface of at least one cell adhesion promoter or cell adhesion inhibitor, which region is spatially related td the transducer so that a cell adhering to said region may be stimulated or detected by said transducer.

10. The biosensor of claim 9, wherein said cell adhesion promoter contains a terminal group selected from the group consisting of —NHCH₂CH₂NHCH₂CH₂NH₂, —NHCH₂CH₂NH₂, 11-aminoundecyl, 3-aminopropyl, 3-(1-aminopropoxy)-3,3-dimethyl-1-propenyl, 6-(aminohexyl)propyl, N-(2-aminoethyl)-3-aminopropyl, —(CH₂)₃—NH—(CH₂)₃—NH—(CH₂)₃, Gly-Arg-Gly-Asp-Tyr-, and Gly-Tyr-Ile-Gly-Ser-Arg-Tyr.

11. The biosensor of claim. 9, wherein said cell adhesion inhibitor is selected from the group consisting of tridecafluoro-1,1,2,2-tetrahydrooctyl)-1-dimethylchlorosilane, tridecafluoro-1,1,2,2-tetrahydrooctyl)-1-trichlorosilane, tridecafluoro-1,1,2,2-tetrahydrooctyl)-1-methyldichlorosilane, tridecafluoro-1,1,2,2-tetrahydrooctyl)-1-triethoxysilane, (3,3,3-trifluoropropyl)trichlorosilane (3,3,3-triflurorpropyl)methyldichlorosilane, (3,3,3-triflurorpropyl)-dimethylchlorosilane, (3,3,3-trifluoropropyl)methyldimethoxysilane, (3,3,3-trifluoropropyl)trimethoxysilane, (heptafluoroisopropoxy) propylmethyldichlorosilane, (3-pentafluorophenylpropyl) dimethylchlorosilane, polyethylene glycols, silanes having a branched or unbranched C₃-C₄₀ alkyl terminus, phenyl groups, and inhibitory biological macromolecules.

12. The biosensor of claim 1, wherein a gigaohm seal is provided between the cell and the substrate.

13. The biosensor of claim 1, wherein a self-assembled monolayer is provided on the substrate in a predefined pattern, and the neuron is provided thereon.

14. The biosensor of claim 13, wherein a cell-repulsive surface is provided at the periphery of the self-assembled monolayer.

15. The biosensor of claim 13, wherein the self-assembled monolayer is composed of trimethoxysilylpropyl diethylene tetraamine (DETA).

16. The biosensor of claim 1, wherein said cell is a hippocampal neuron.

17. The biosensor of claim 1, wherein said transducer is capable of stimulating said electrically excitable cell.

18. The biosensor of claim 1, wherein the transducer is formed in the substrate.

19. A biosensor comprising:
a substrate;
first and second neurons provided on at least a portion of the substrate, said neurons each having a predefined polarity;
a first transducer adjacent one of said neurons and capable of detecting a signal therein; and
a second transducer adjacent one of said neurons and capable of stimulating or detecting a signal therein, said neurons being in synaptic relationship so that a signal established in one of the neurons is attenuated by the other neuron.

20. The biosensor of claim 19, further comprising a stimulator adjacent one of said first and second neurons, which upon stimulation is capable of affecting a signal established therein.

21. The biosensor of claim 19, wherein said first and second transducers are microelectrodes.

22. The biosensor of claim 19, wherein said first and second transducers are field effect transistors.

23. A method of detecting a bioeffecting substance in a test sample, comprising:
(a) providing said test sample and a biosensor as in claim 1, wherein at least one cell of said biosensor produces a detectable response to said bioeffecting substance;
(b) contacting the test sample with the biosensor;
(c) monitoring, with a transducer of the biosensor, a signal produced by said at least one cell in response to contacting said cell with the test sample; and
(d) correlating said signal to the presence or absence of said bioeffecting substance in the test sample.

24. The method of claim 23, wherein the biosensor comprises at least two transducers.
